(19) United States
(12) Patent Application Publication
(10) Pub. No.: US 2010/0028960 A1
(43) Pub. Date: Feb. 4, 2010

(54) Preparation of Precisely Controlled Thin Film Nanocomposite of Carbon Nanotubes and Biomaterials

(75) Inventors: Virginia A. Davis, Auburn, AL (US); Aleksandr L. Simonian, Auburn, AL (US); Dhriti Nepal, Auburn, AL (US); Shankar Balasubramanian, San Diego, CA (US)

Correspondence Address: ANDRUS, SCEALES, STARKE & SAWALL, LLP, 100 EAST WISCONSIN AVENUE, SUITE 1100, MILWAUKEE, WI, 53202, US

(73) Assignee: Auburn University, Auburn, AL (US)

(21) Appl. No.: 12/261,892

(22) Filed: Oct. 30, 2008

Related U.S. Application Data
(60) Provisional application No. 61/000,938, filed on Oct. 30, 2007.

(51) Int. Cl. C12P 9/00; B32B 9/00; B05D 1/36; A61L 2/00

(52) U.S. Cl. 435/131; 428/336; 427/414; 422/28

(57) ABSTRACT

Disclosed are nanocomposite materials comprising multiple layers of biomolecules bound to aligned carbon nanotubes. The thickness of each of the layers may be precisely controlled using a layer-by-layer assembly technique.

### CROSS-REFERENCE TO RELATED APPLICATION

The present application claims the benefit of priority under 35 U.S.C. §119(e) to U.S. provisional application No. 61/000,938, filed on Oct. 30, 2007, the content of which is incorporated herein by reference in its entirety.

### STATEMENT REGARDING FEDERALLY SPONSORED RESEARCH OR DEVELOPMENT

This invention was made with U.S. Government Support from the following agencies: National Science Foundation (NSF) Grant No. CTS-0330189; and United States Department of Agriculture-Cooperative State Reseach, Education, and Extension Service (USDA-CSREES) Grant No. 2006-34394-16953. The U.S. Government has certain rights in the invention.

### BACKGROUND

The present invention relates generally to nanocomposite materials that include biomaterials and carbon nanotubes such as single-walled carbon nanotubes (SWNT), double-walled carbon nanotubes (DWNT), and few-walled carbon nanotubes (FWNT). The nanocomposite materials typically are prepared by a layer-by-layer technique.

Carbon nanotubes exhibit desirable properties that make them potentially useful in numerous applications. For example, CNTs may exhibit high strength and conductivity. These properties make CNTs potentially useful in many applications including material science applications and electronics.

Carbon nanotubes may be combined with other organic or inorganic materials to form nanocomposites. In particular, CNTs have been combined with biomolecules such as polypeptides and polynucleotides to form nanocomposites. (See, e.g., Munge et al., Anal. Chem. Jul. 15, 2005;77(14):4662-6; Liu et al., J. Nanosci. Nanotechnol. 2006 April;6(4):948-53; Ishibashi et al., Chem. Phys. Lett. Feb. 26, 2006;419(4-6):574-577). However, further nanocomposites of CNTs and specific biomaterials are desirable. In particular, nanocomposites having anti-microbial activity are desirable, for example with respect to bacteria. Furthermore, nanocomposites having decontaminating activity also are desirable, for example with respect to organophosphorus chemicals. In addition, nanocomposites of CNTs and biomolecules that have suitable hardness, Young's modulus, and controlled morphology (e.g., with respect to thickness) also are desirable as are new methods for preparing such nanocomposites.

### SUMMARY

Disclosed are nanocomposite materials such as nanocomposite films and coatings. The films and coatings may be free standing or may be present on solid substrates. The nanocomposite materials disclosed herein typically include multiple layers of biomolecules bound to aligned carbon nanotubes (e.g., aligned SWNT, aligned DWNT, or aligned FWNT). The alignment of the carbon nanotubes and the thickness of the multiple layers in the disclosed nanocomposite materials are precisely controlled. In some embodiments, the multiple layers individually have an average thickness of 1-2 times the average diameter of the carbon nanotubes (e.g., where multiple layers of SWNT bound to selected biomolecules individually have an average thickness of about 1-2 nm (and in some embodiments about 1.6 nm (±0.03 nm)). In further embodiments, the multiple layers individually have an average thickness that is proportional to the average diameter of the carbon nanotubes.

Suitable biomolecules may include, but are not limited to, polypeptides (or proteins), polynucleotides (e.g., DNA), and mixtures thereof. In preferable embodiments, the biomolecule has anti-bacterial activity and the nanocomposite materials incorporating the biomolecule also have anti-bacterial activity. Suitable anti-bacterial polypeptides include lysozyme. In other embodiments, the biomolecule has decontaminating activity, for example, with respect to organophosphorus chemicals. Suitable decontaminating polypeptides include, but are not limited to, organophosphorus (OP) hydrolyzing enzymes (OPH=Organophosphorus Acid Hydrolase; OPAA=Organophosphorus Acid Anhydrolase), and nanocomposite materials that incorporate OPH and OPAA may exhibit organophosphorus hydrolase activity

The nanocomposite material disclosed herein may include films having multiple layers in which the CNTs incorporated therein are aligned. The multiple layers may include: (a) at least a first layer wherein the nanotubes are aligned in a first direction; and (b) at least a second layer adjacent to the first layer wherein the nanotubes are aligned in a second direction. In some embodiments, the first direction and the second direction are the same (i.e., where the CNTs in the first and second layer are parallel). In other embodiments, the first direction and the second direction may be non-parallel, at a 45° angle, or perpendicular (i.e., wherein the CNTs in the first and second layer are perpendicular). In further embodiments, the nanotubes of each layer of the multiple layers may be aligned perpendicularly to the nanotubes of each adjacent layer (i.e., where the multiple layers are alternating perpendicular layers).

The nanocomposite material disclosed herein may include films have multiple adjacent layers of alternating surface charges. In some embodiments, the multiple layers include: (a) at least a first layer comprising positively-charged biomolecules (e.g., positively-charged polypeptides) bound to single wall carbon nanotubes; and (b) at least a second layer adjacent to the first layer, the second layer comprising negatively-charged biomolecules or polymers bound to single wall carbon nanotubes. In other embodiments, the multiple layers include: (a) at least a first layer comprising negatively-charged biomolecules (e.g., negatively-charged polypeptides) bound to single wall carbon nanotubes; and (b) at least a second layer adjacent to the first layer, the second layer comprising positively-charged biomolecules or polymers bound to single wall carbon nanotubes. The nanocomposite material disclosed may include films or coatings having a desirable thickness. For example, a desirable thickness may be obtained by applying a selected number of layers of CNTs bound to biomolecules to a solid substrate. In some embodiments, the films or coatings have a thickness of at least about 5 nm (preferably at least about 10 nm, more preferably at least about 50 nm, even more preferably at least about 100). The films or coatings may comprise any suitable number of layers (e.g., at least about 10, 20, 30, 40, 50, 100, 200, or more layers). The nanocomposite material disclosed herein may include films or coatings having a desirable hardness. In some embodiments, the films or coatings have a hardness of at least about 0.5 GPa (preferably at least about 1 GPa, more preferably at least about 2 GPa).

The nanocomposite material disclosed herein may include films or coatings having a desirable Young's modulus. In some embodiments, the films or coating have a Young's modulus of at least about 10 GPa (preferably at least about 20 GPa, more preferably at least about 30 GPa).

The disclosed nanocomposite material may be freestanding or may be applied as a film or coating to a solid substrate. In some embodiments, contemplated methods for preparing a coated substrate include: (a) coating the substrate with a first layer, the first layer comprising biomolecules bound to carbon nanotubes, and aligning the carbon nanotubes by shear force, wherein the first layer preferably has an average thickness of 1-2 times the average diameter of the carbon nanotubes (and in some embodiments of SWNT, an average thickness of 1-2 nm or about 1.6 nm (±0.03 nm)), and the first layer has a surface charge that is opposite to a surface charge for the substrate; (b) subsequently coating the substrate with a second layer, the second layer comprising biomolecules bound to carbon nanotubes, and aligning the carbon nanotubes by shear force, wherein the second layer preferably has a thickness of 1-2 times the average diameter of the carbon nanotubes (and in some embodiments of SWNT, an average thickness of about 1-2 nm or about 1.6 nm (±0.03 nm)), and the second layer has a surface charge that is opposite to the surface charge for the first layer; and (c) repeating (a) and (b) for a suitable number of times to provide a coating having a thickness of at least about 5 nm (preferably at least about 10 nm, more preferably at least about 50 nm, even more preferably at least about 100). As contemplated herein, suitable biomolecules for the methods include anti-bacterial polypeptides (e.g., lysozyme) and decontaminating polypeptides (e.g., organophosphorus hydrolases).

Also contemplated are methods of killing microorganisms and methods of inhibiting the growth of microorganisms such as bacteria. In some embodiments, the methods may include contacting bacteria with a carbon nanocomposite film comprising multiple layers, wherein the multiple layers comprise anti-bacterial polypeptides (e.g., lysozyme) bound to aligned carbon nanotubes. Preferably, the multiple layers individually have an average thickness of 1-2 times the average diameter of the carbon nanotubes. In some embodiments of SWNT, the individual layers have an average thickness of about 1-2 nm or about 1.6 nm (±0.03 nm).

Also contemplated are methods of decontaminating a surface that contains organophosphorus chemicals and methods of preventing contamination of a surface with organophosphorus chemicals. In some embodiments, the methods may include hydrolyzing the organophosphorus compounds by contacting the compounds with a carbon nanocomposite film comprising multiple layers, wherein the multiple layers comprise one or more different organophosphorus hydrolase polypeptides or proteins bound to aligned carbon nanotubes and the layer hydrozes, inactivates, or destroys organophosphorus compounds. Preferably, the multiple layers individually have an average thickness of 1-2 times the average diameter of the carbon nanotubes. In some embodiments of SWNT, the individual layers have an average thickness of about 1-2 nm or about 1.6 nm (±0.03 nm).

### BRIEF DESCRIPTION OF THE FIGURES

FIG. 1. (a) Turbidimetric assay of LSZ and LSZ-SWNT conjugate in solution against M. lysodeikticus. (b) Rate of M. lysodeikticus lysis reaction (regression line is fit to the linear portion of experimental data points in (a) using first-order kinetics).

FIG. 2. (a) UV-vis-NIR absorbance spectra of LBL assembly of LSZ-SWNT/DNA-SWNT (concentration of SWNT in dispersion ˜25 mg/L). The inset magnifies the van Hove transitions of metallic and semiconducting SWNT. (b) Comparison of UV-vis accumulation curves for absorbance at 510 nm and ellipsometry thickness measurement of the LBL assembly. (c) AFM image of DNA-SWNT dried dispersion. (d) Schematic diagram of LBL assembly of LSZ-SWNT and DNA-SWNT.

FIG. 3. SEM images of LBL assembly of LSZ-SWNT/DNA-SWNT of the (a) 8th layer and (b) 68^{th} layer. (c) Raman spectra of the assembly (8th layer) showing D-band to G-band recorded at various angles between the polarization of laser excitation and SWNT alignment direction using 514 nm laser d) Raman mapping collected at 10×10 μm area (8th layer) showing D-band and G-band. The scale bars in (a) and (b) represent 200 nm.

FIG. 4. UV-vis-NIR absorbance spectra of LBL assembly of LSZ-SWNT/DNA-SWNT obtained from dispersion of SWNT at higher concentration (45 mg/L). Blue represents DNA-SWNT and red represents LSZ-SWNT (a) without NaCl, (b) with addition of NaCl (10 mM). The insets in (a) and (b) magnifies the van Hove transitions of metallic and semiconducting SWNTs. (c) Surface plasmon resonance of in situ thin film deposition showing the surface coverage. Comparison of UV-vis accumulation curves for absorbance at 510 nm and ellipsometry thickness measurements of the LBL assembly from 45 mg/L SWNT dispersions (d) without NaCl and (e) with addition of NaCl (10 mM). (f) and (g) are SEM images of the surface of the film (68^{th} layer) without NaCl and with NaCl respectively. The scale bars in (f) and (g) represent 200 nm.

FIG. 5. Nanoindentation tests on a 68 layer coating (LSZ-SWNT/DNA-SWNT)₆₈ (a) hardness (b) Young's modulus. The inset in (b) shows plateau region where Young's Modulus was calculated.

FIG. 6. (a) Effect of different layers of LBL coating against M. lysodeikticus in turbidimetric assay. (b) Rate of M. lysodeikticus lysis reaction (Regression line is fit to the linear portion of experimental data in (a) using first-order rate kinetics). SEM image of samples incubated with Staphylococcus aureus at 37° C. for 24 hrs of (c) a clean silicon wafer (control) and (d) LBL assembly at 11th layer (top surface LSZ-SWNT) arrows indicating damaged cells). The scale bars in (c) and (d) represent 1 μm.

FIG. 7. Illustrates the activity of 21 layer LBL coating on the first and sixtieth day by turbidimetric assay

### DETAILED DESCRIPTION

The disclosed subject matter is further described below.

Unless otherwise specified or indicated by context, the terms “a”, “an”, and “the” mean “one or more.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≦10% of the particular term and “substantially” and “significantly” will mean plus or minus >10% of the particular term.

As used herein, the terms “include” and “including” have the same meaning as the terms “comprise” and “comprising.”

The disclosed nanocomposite materials include “carbon nanotubes” (CNTs). “Nanotubes” alternately may be referred to in the art as “nanocylinders,” “nanorods,” or “nanowires.” Carbon nanotubes have a tubular or cylindrical in structure and further are members of the fullerene structural family, which is characterized by linked hexagonal rings and occasional pentagonal or heptagonal rings. Carbon nanotubes are long, thin, hollow cylinders formed by rolling a single layer of graphite. Carbon nanotubes typically have an average diameter (D) that is less than 100 nm and an average persistence length (L) that is at least five times the average diameter (i.e., L≧(5×D)) (preferably an average diameter that is less than 20 nm and an average persistence length that is greater than about 100 nm). In some embodiments, inorganic nanocylinders as contemplated herein have an aspect ratio that is at least about 5 (preferably at least about 10, 20, 50, 100, 500, or even 1000). The carbon nanotubes utilized herein may be single-walled carbon nanotubes (SWNTs), double-walled carbon nanotubes (DWNTs), or few-walled carbon nanotubes (FWNTs).

The disclosed nanocomposite materials include multiple layers of aligned CNTs. The alignment and uniform dispersion of the CNTs within a layer may be quantified by Raman spectroscopy as disclosed herein (e.g., by calculating the Raman ratio G⁰/G⁹⁰) or as understood in the art, for example, using the Fraser fraction, f where f=(R−1 )/(R+4), and R, the alignment ratio, is the Raman intensity ratio between the parallel and perpendicular orientations of the nanotube aggregates. (See, e.g., U.S. Pat. No. 7,125,502, which content is incorporated herein by reference in its entirety). In some embodiments, the Raman ratio for a layer is at least about 5 (or at least about 6 or at least about 7).

The nanocomposite materials disclosed herein may include films or coating prepared using a layer-by-layer (LBL) assembly technique in which each layer is prepared by dipping the film or coating (or the film or coating as attached to a solid substrate) into a solution having an opposite surface charge from a previous applied layer (or an opposite surface charge than the solid substrate for the first applied layer). The applied solution comprises carbon nanotubes and the selected biomolecules. After a layer is applied, the carbon nanotubes and the biomolecules bound thereto may be dispersed and aligned in the layer using any suitable technique, including but not limited to application of shear force to the layer (e.g., by blowing air across the layer). A subsequently applied layer may have an alternate surface charge than a previously applied layer. Further, the carbon nanotubes and the biomolecules bound thereto of a subsequently applied layer may be aligned in the same or in a different direction than the carbon nanotubes and the biomolecules bound thereto of the previously applied layer. For example, in some embodiments alternating layers may have carbon nanotubes and the biomolecules bound thereto aligned parallel or perpendicular. The thickness of each layer also may be precisely controlled. For example, the thickness of each layer may approximate the diameter of a single carbon nanotube (e.g., a SWNT) bound to the selected biomolecules. In some embodiments, the multiple layers individually have an average thickness of 1-2 times (×) the average diameter of the carbon nanotubes (or an average thickness of 1.1-1.9×the average diameter of the carbon nanotubes, an average thickness of 1.2-1.8×the average diameter of the carbon nanotubes, an average thickness of 1.3-1.7×the average diameter of the carbon nanotubes, an average thickness of 1.4-1.6×the average diameter of the carbon nanotubes, or an average thickness of 1.5×the average diameter of the carbon nanotubes. In some embodiments of SWNT, the thickness of each layer is approximately 1-2 nm or about 1.6 nm (±0.03 nm)). Layer-by-layer assembly and alignment of carbon nanotubes and the biomolecules bound thereto may be monitored and confirmed in situ by using techniques in the art, including but not limited to near infrared radiation (NIR), surface plasmon resonance (SPR), cyclic voltammetry (CV), ellipsometry, and scanning electron microscopy (SEM).

The disclosed nanocomposite materials include aligned carbon nanotubes and biomolecules bound thereto. Suitable biomolecules may include, but are not limited to, polypeptides, polynucleotides, and mixtures thereof that are naturally-occurring. A “naturally-occurring polypeptide” refers to a chain of amino acids that occurs in nature. Suitable polypeptides may include enzymes. The term “polypeptides” as utilized herein may include multi-subunit polypeptides or proteins. A “naturally-occurring” nucleic acid molecule refers to a DNA or RNA molecule having a nucleotide sequence that occurs in nature (e.g., a DNA or RNA molecule encoding a naturally-occurring protein or a fragment thereof). Suitable polypeptides may include anti-microbial polypeptides (e.g., anti-bacterial, anti-fungal, and/or anti-viral polypeptides). Suitable anti-bacterial polypeptides may include lysozyme or other anti-bacterial polypeptides as understood in the art (see, e.g., Lata S. et al., BMC Bioinformatics Jul. 23, 2007;8:263, which is incorporated by reference herein in its entirety). Polypeptides may include polypeptides having organophosphorus hydrolase activity (e.g., for decontaminating a surface containing organophosphorus chemicals). Organophosphorus hydrolase (OPH, EC 8.1.3.2 or “phosphotriesterase”) and Organophosphorus Acid Anhydrolase (OPAA, EC 3.1.8.1) are enzymes that catalyzes the hydrolysis of organophosphorus pesticides and nerve agents and can be utilized to decontaminate a surface containing such pesticides or nerve agents. Furthermore, suitable biomolecules may include polynucleotides, and suitable polynucleotides may include genes or gene fragments.

The disclosed nanocomposite materials include aligned carbon nanotubes and biomolecules bound thereto. In some embodiments, the disclosed biomolecules may bind to the carbon nanotubes non-covalently based on surface chemistries. Optionally, the carbon nanotubes may be functionalized to facilitate covalent binding or additional non-covalent interactions with the biomolecules. Suitable functional groups may include carboxyl groups, thioalkyl groups, hydroxyl groups, alkyl groups, and the like.

The nanocomposite materials disclosed herein which include aligned carbon nanotubes and biomolecules bound thereto provide a fundamental improvement in products and articles of manufacture that rely on dispersed, aligned carbon nanotubes. Some of the articles of manufacture include, but are not limited to, composite materials with chemical, electrical, mechanical, or electromagnetic properties derived in part from the carbon nanotubes and biomolecules bound thereto. The dispersion of aligned carbon nanotubes and biomolecules bound thereto as contemplated herein may enable better properties for applications including but not limited to biologically-compatible coatings, objects and devices that are inserted or implanted into living organisms, chemical, physical, and electronic sensors, and fiber material for clothing or other structures.

### Examples

The following Examples are illustrative and are not intended to limit the scope of the claimed subject matter. Reference is made to Nepal et al., “Strong Antimicrobial Coatings: Single-Walled Carbon Nanotubes Armored with Biopolymers,” Nano Letters 2008 8(7):1896-1901, (the content of which is incorporated by reference herein in its entirety).

Abstract

Large scale biomimetic single-walled carbon nanotube (SWNT) coatings with significant antimicrobial activity, high Young's Modulus, and controlled morphology were fabricated using layer-by-layer assembly. Thickness was controlled within 1.6 nm and SWNT orientation was controlled using a directed air stream. This unique blend of multifunctionality and vertical and lateral control of a bottom-up assembly process is a significant advancement in developing macroscale assemblies with the combined attributes of SWNTs and natural materials.

Background, Results, and Discussion

Concern about the spread of infections through contact with contaminated surfaces was once limited to specific groups of people including astronauts who are subject to confined living spaces and the virulence-enhancing effects of space flight (see Wilson, J. W et al., Proceedings of the National Academy of Sciences 2007, 104, 16299-16304) and people requiring surgery or implantable devices (see Darouiche, R. O. The New England Journal of Internal Medicine 2004, 350, 1422-1429). More recently, there has been growing concern about the role of contaminated surfaces in the spread of infections such as severe acute respiratory syndrome (SARS) (see Cheng, V. C. C. et al., Proceedings of the National Academy of Sciences 2007, 20, 660-694; and Chu, C.-M. et al., Emerging Infectious Diseases 2005, 11, 1882-1886) and staphylococcus aureus, particularly methicillin-resistant staphylococcus aureus (MRSA) (see Klevens, R. M. et al., The Journal of the American Medical Association 2007, 298, 1763-1771). Antimicrobial surfaces are therefore desirable not only for the aerospace, defense and medical industries, but also for the consumer product and public transportation industries. We have used layer-by-layer assembly to produce coatings that combine the strength of single-walled carbon nanotubes (SWNTs) with the antimicrobial activity of lysozyme (LSZ).

LSZ, a key member of ova-antimicrobials, is a powerful natural antibacterial protein (see Jolles, J. et al., Mol. Cell. Biochem. 1984, 63, 165-189). It is in the class of enzymes which lyse the cell walls of gram-positive bacteria by hydrolyzing the β-1,4 linkage between N-acetylmuramic acid (NAM) and N-acetylglucosamine (NAG) of gigantic polymers in the peptidoglycan (murein) (see Proctor, V. A. et al., Critical Reviews in Food Science and Nutrition 1988, 26, 359-395; and Losso, J. et al., Natural Food Antimicrobial Systems; Naidu, A. S., Ed.; CRS Press: 2000, p 185-210). Unlike many antimicrobials, LSZ has both enzymatic and non-enzymatic activity in both its native and denatured states and is useful even in processes which require heat treatment. The potential use of LSZ as an antimicrobial agent in pharmaceuticals, food preservatives and packaging is an active area of research, (see Proctor, V. A. et al., Critical Reviews in Food Science and Nutrition 1988, 26, 359-395; and Losso, J. et al., Natural Food Antimicrobial Systems; Naidu, A. S., Ed.; CRS Press: 2000, p 185-210), but the effective use of LSZ requires incorporating it with a more mechanically robust material. SWNTs are well known for exceptional combination of mechanical, electrical, thermal and optical properties (see Huang, J. Y. et al., Nature 2006, 439, 281-281; and O'Connell, M. J., et al., Science 2002, 297, 593-596; and Nepal, D. et al., Functionalization of Carbon Nanotubes Geckeler, Kurt E., Rosenberg, E., Eds.; American Scientific Publishers: Valencia, 2006, p 57-79). However, the efficient transfer of SWNTs' inherent nanoscale properties to macroscopic structures and devices has been an ongoing research challenge comprised of three main issues: SWNT dispersion, controlled assembly, and efficient load transfer. There has been growing interest in using biological materials to stabilize dispersions of pristine SWNTs. DNA enables much higher concentrations dispersions of individual and small bundles of SWNTs (see Nepal, D. et al., Functionalization of Carbon Nanotubes Geckeler, Kurt E., Rosenberg, E., Eds.; American Scientific Publishers: Valencia, 2006, p 57-79; and Nepal, D. et al., Biomacromolecules 2005, 6 2919 -2922) than any other known material besides superacids (see Davis, V. A. et al., Macromolecules 2004, 37, 154-160; and Rai, P. K. et al., J. Am. Chem. Soc. 2006, 128, 591-595). DNA-SWNT dispersions have even been used to produce liquid crystalline dispersions for solution spinning (see Barisci, J. N. et al., Advanced Functional Materials 2004, 14, 133-138). Similarly, favorable intermolecular interactions enable dispersion of individual and small bundles of SWNTs in proteins such as LSZ (see Nepal, D. et al., Functionalization of Carbon Nanotubes Geckeler, Kurt E., Rosenberg, E., Eds.; American Scientific Publishers: Valencia, 2006, p 57-79; Nepal, D. et al., Small 2006, 2, 406-412; and Nepal, D. et al., Small 2007, 3, 1259-1265). In this research, the strong columbic interactions between DNA and LSZ were exploited in the layer-by-layer (LBL) assembly of DNA-SWNT and LSZ-SWNT dispersions (see Hammond, P. T. Current Opinion in Colloid & Interface Science 1999, 4, 430-442; Tang, Z. et al., Advanced Materials 2006, 18, 3203-3224; and Jiang, C. et al., Advanced Materials 2006, 18, 829-840).

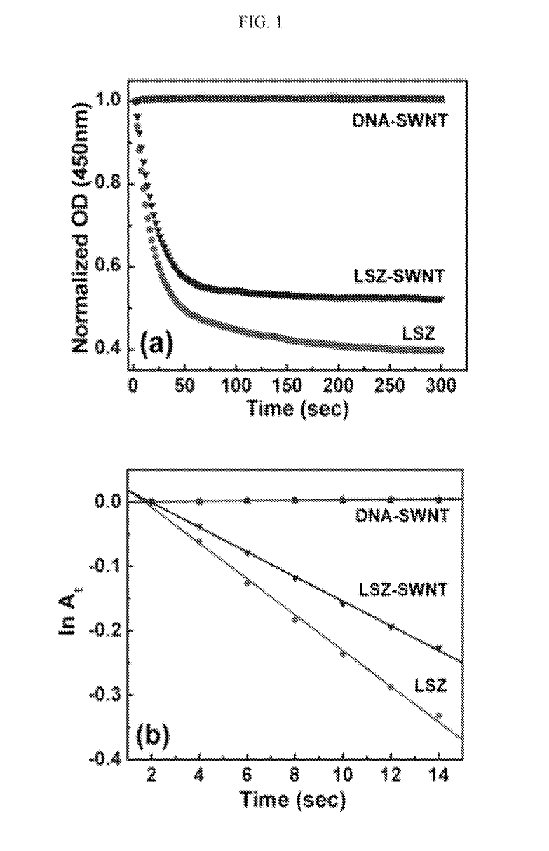

The enzymatic activity of LSZ in the SWNT dispersions was determined by measuring the rate of lysis of gram-positive Micrococcus lysodeikticus intact cells (FIG. 1). The responses from the turbidimetric assay were modeled with first-order kinetics typically used to quantify exponential death of microorganisms (FIG. 1b). The analysis shows that LSZ-SWNT dispersions clear approximately 55% of turbidity (optical density at 450 nm) within five minutes compared to 60% for the LSZ dispersion. The decrease in optical density due to cellular lysis confirms that secondary structure of LSZ in LSZ-SWNT conjugate is well preserved Nepal, D.; Geckeler, K. E. Small 2006, 2, 406-412 as required for enzyme activity. In contrast, the DNA-SWNT dispersions showed no reduction in turbidity, suggesting that the SWNTs did not contribute to cell lysis.

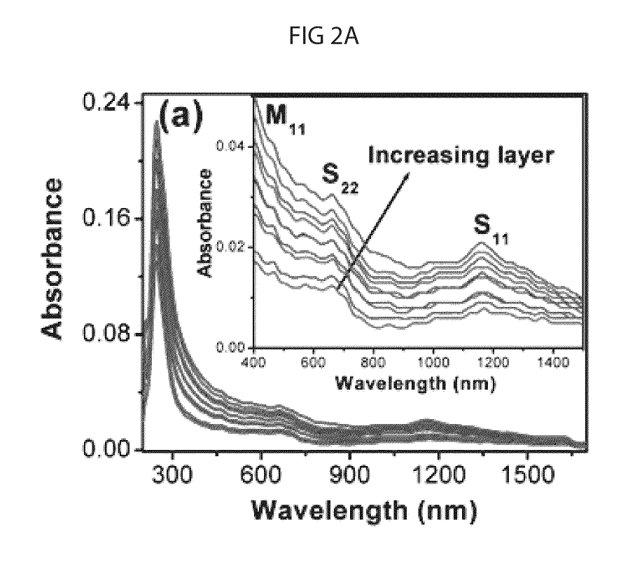

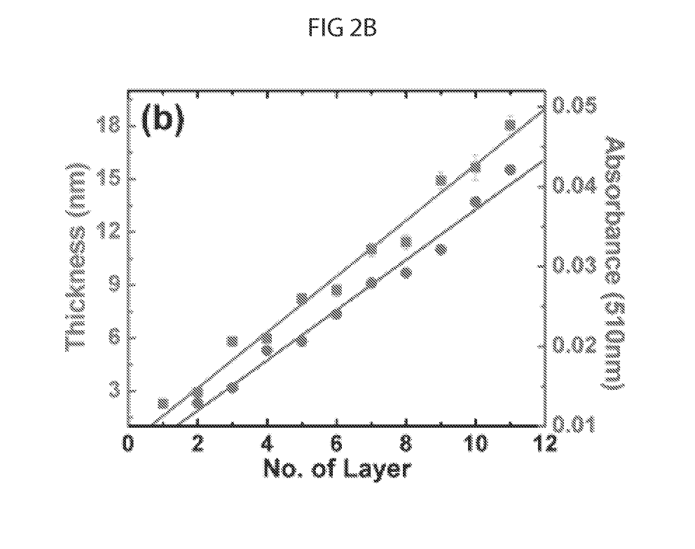

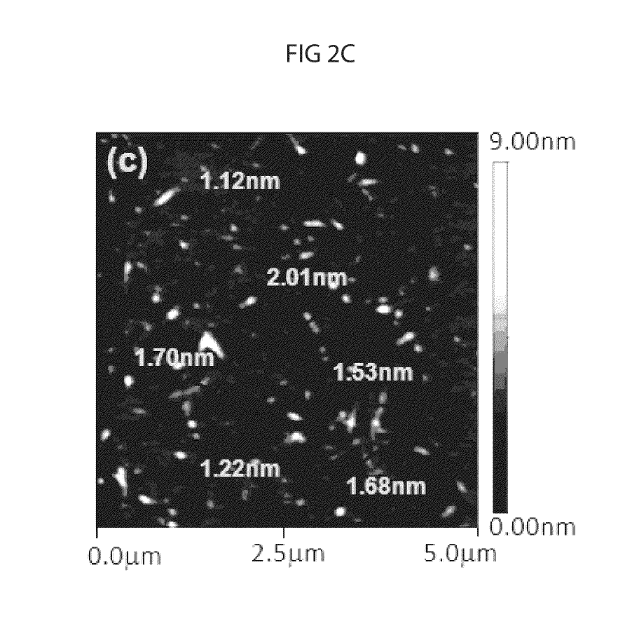

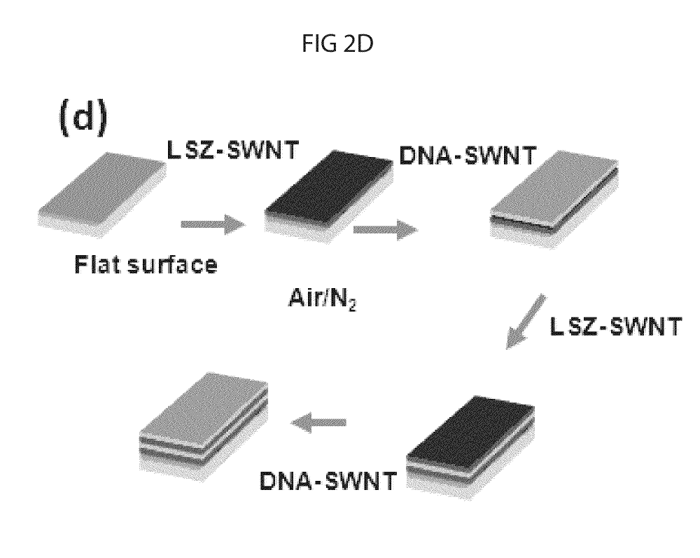

Zeta potential measurements confirmed that the cationic and anionic nature of LSZ-SWNT (+22 mV) and DNA-SWNT dispersions (−30 mV) provided an excellent platform for strong electrostatic interaction between LSZ-SWNT and DNA-SWNT coatings [(LSZ-SWNT)-(DNA-SWNT)]ₙ. Secondary forces vital in DNA-protein interactions in biological systems, including van der Waals and π-π attractions, were also likely to have played a significant role in inter-layer adhesion. UV-vis-NIR absorption spectroscopy, ellipsometry and surface plasmon resonance (SPR) were used in concert to monitor the growth of the LBL coatings on a variety substrates including silicon, gold, glass and mica (supporting information). Absorbance spectroscopy (FIG. 2a) showed well-resolved van Hove transitions of metallic (M₁₁) and semiconducting SWNTs (S₁₁ and S₂₂), indicating that the SWNTs retained their electronic structure in the matrix and were predominantly dispersed as individual SWNTs (see O'Connell, M. J. et al., Science 2002, 297, 593-596). The uniform increase of UV-vis-NIR absorbance from each deposition cycle revealed that film growth was linear and uniform. Moreover, ellipsometry showed a 1.6 nm (±0.03 nm) increase in thickness per layer (FIG. 2b); a value consistent with the previously reported diameter of individual DNA-SWNT adducts (see Zheng, M. et al., Nature Materials 2003, 2, 338-342). Atomic force microscopy (AFM) provided further verification of deposition of individual SWNTs; the average diameters of the DNA-SWNT adducts (FIG. 2c) was 1.6 nm. This extremely fine control of assembly process is the direct result of the quality of the initial dispersion.

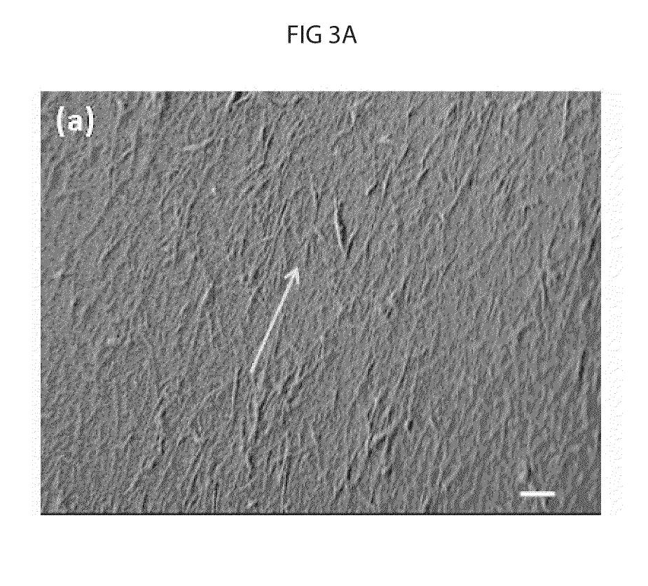

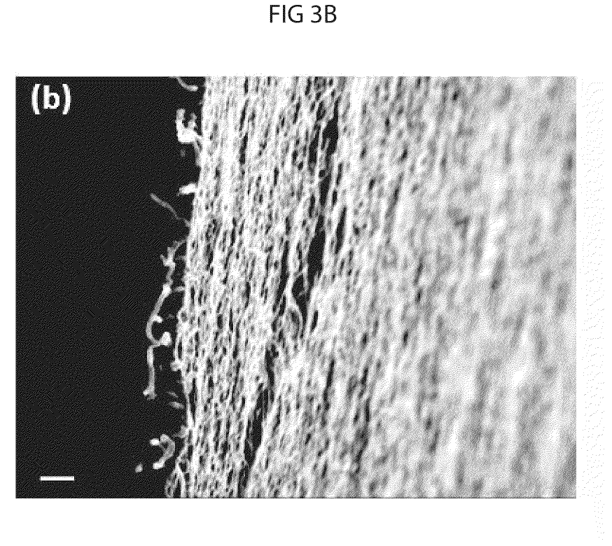

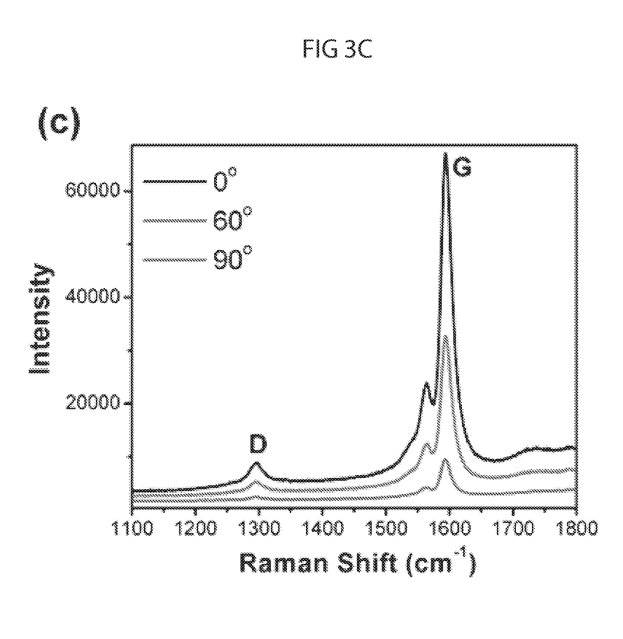

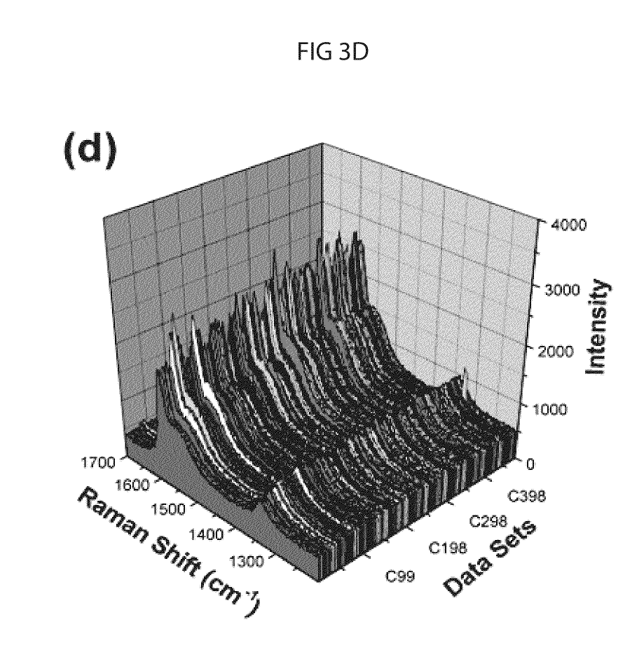

SWNT orientation within each layer was achieved by applying a directed air stream between each deposition step. This step decreased the time required for assembly by eliminating the need for the rinsing step inherent in many LBL processes. Furthermore, the air stream enabled shear alignment of SWNTs within each individual layer generating the possibility to create coatings where each layer has a distinct orientation. FIGS. 3a and 3b show SEM images of the aligned 8^{th} and 68^{th} layers, respectively. Uniform deposition and alignment were further confirmed by Raman spectroscopy. Raman mapping was conducted to evaluate the spatial distribution of SWNT on the surface; G band intensities across a wide area (10 μm²) are almost uniform showing that the SWNTs were uniformly spaced. It is well established that Raman intensity of SWNTs is maximal when exciting light polarization is along the nanotubes axis (see Jorio, A. et al., Physical Review Letters 2000, 85, 2617; Ericson, L. M. et al., Science 2004, 305, 1447-1450; and Chae, H. G. et al., Polymer 2006, 47, 3494-3504). When the air stream was applied in random directions, no change in Raman intensity was observed indicating an isotropic orientation. However, directing the air stream in one direction resulted in a significant degree of alignment and a Raman ratio (G⁰/G⁹⁰) of 7 (FIG. 3c). The ability to align the SWNTs using a directed air stream is due to SWNTs rigid rod behavior in solution (see Doi, M. et al., The Theory of polymer Dynamics; Oxford University Press: Oxford, 1986; and Duggal, R. et al., Physical Review Letters 2006, 96). Shear induced alignment of SWNTs has also been observed in films and fibers (see Shim, B. S. et al., Langmuir 2005 21, 9381-9385; Hedberg, J. et al., Applied Physics Letters 2005, 86, 143111-3; and Davis, V. A. et al., Nanoengineering of Structural, Functional and Smart Materials; Schulz, M. J., Kelkar, A., Sundaresan, M. J., Eds.; CRC Press: Boca Raton, 2005).

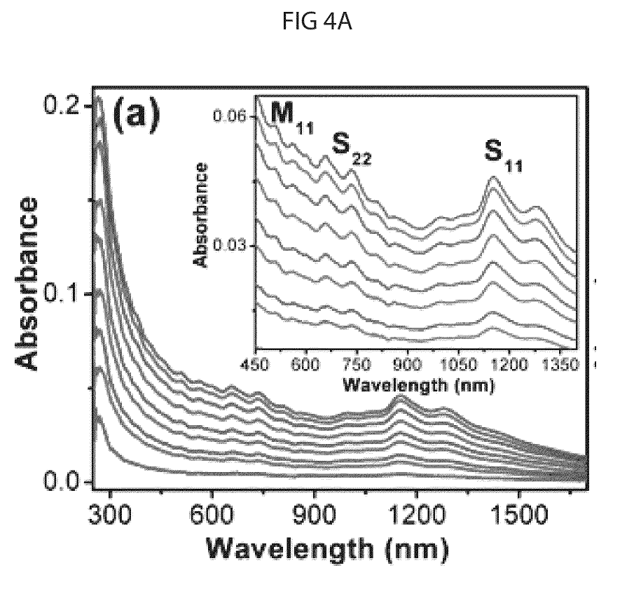

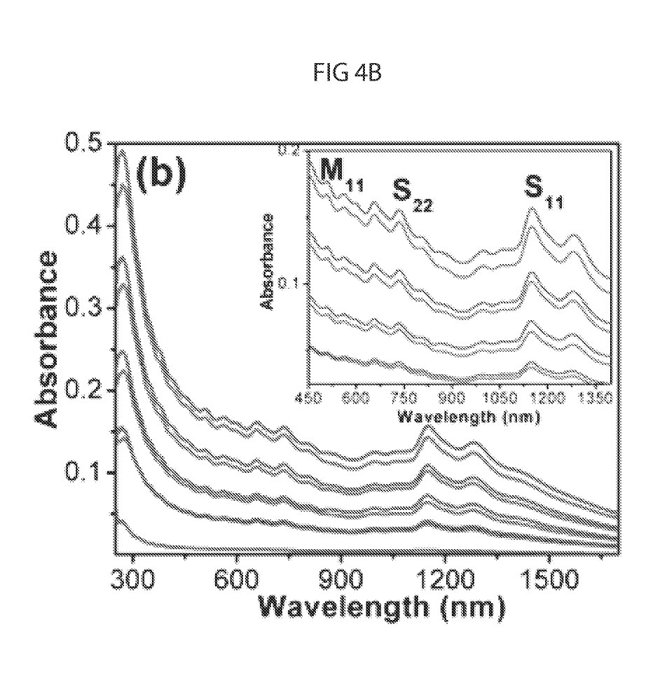

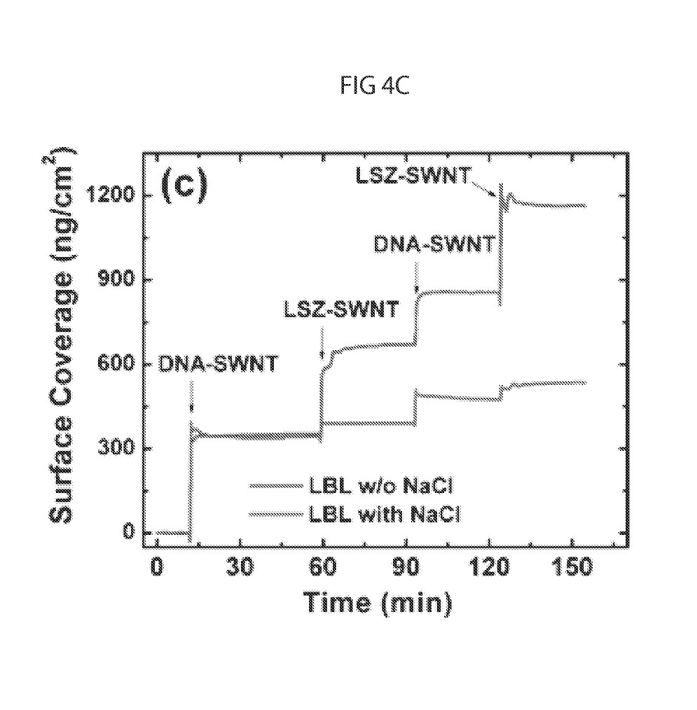

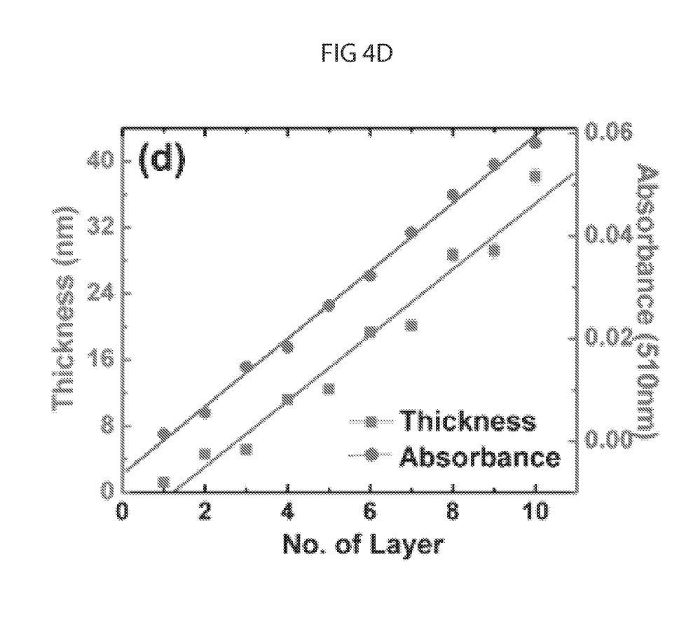

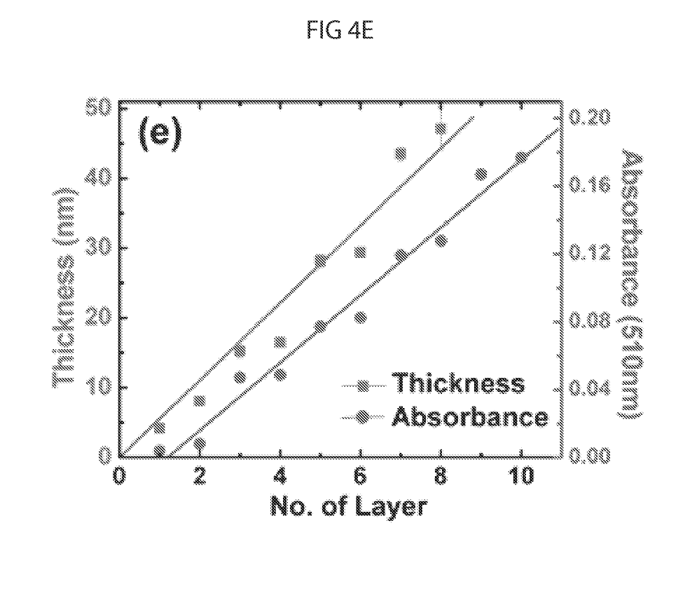

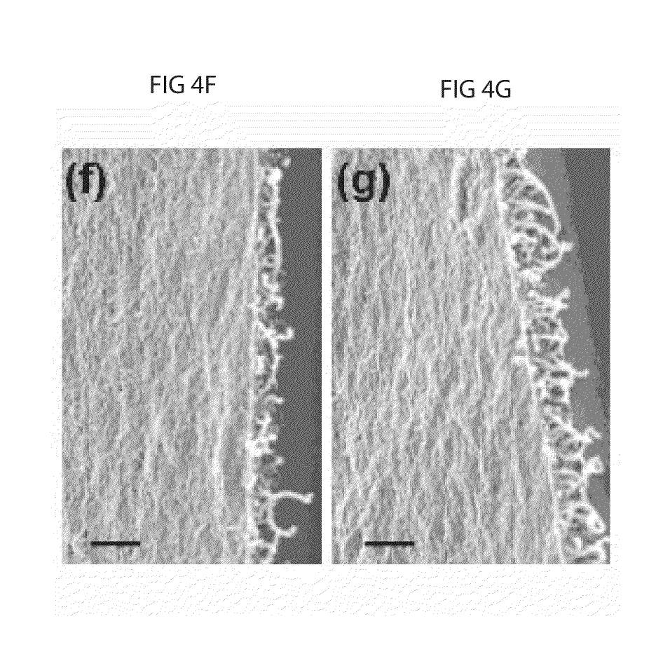

In order to better understand the LBL process, dispersions with different SWNT concentrations were produced with and without added electrolyte. SWNT concentration strongly influenced coating thickness. FIG. 4 shows the UV-vis-NIR absorbance of LBL assembly from the 45 mg/L SWNT dispersion. The increasing intensity corresponds to increased SWNT concentration after the deposition of each layer (FIG. 4a). The presence of clear van Hove peaks suggests that the SWNTs were predominantly individuals, but SEM revealed some small aggregates non-parallel overlapping SWNTs. Ellipsometry showed (FIG. 4d) that increasing SWNT concentration from 25 mg/L to 45 mg/L increased the average layer thickness from 1.6 nm to 3.0 nm. The increased layer thickness at higher concentrations is believed to be largely due to SWNTs overlapping during the deposition process, and perhaps an increase in the number of small bundles in the dispersion.

The addition of electrolyte also had an effect on the assembly process. FIG. 4b shows the absorbance spectroscopy of LBL growth prepared in LSZ-SWNT and DNA-SWNT dispersions containing 45 mg/L SWNT and 10 mM NaCl. On each LSZ-SWNT layer, the rate of growth of absorbance is faster than that with DNA-SWNT. This corroborates with ellipsometry results (FIG. 4e) and is further confirmed by surface plasmon resonance (SPR) spectroscopy, a surface sensitive technique which unambiguously demonstrates the effect of salt in LBL film assembly (FIG. 4c). The rapid increase in SPR response provides strong evidence for electrostatic interactions between oppositely charged SWNT-bioadducts. SPR response increases smoothly over each layer indicating progressive assembly; surface coverage calculations indicate that more nanotubes were deposited in the presence of 10 mM NaCl. The influence of added salt agrees well with reported values for similar SWNT-polyelectrolyte films and coatings (see Hofmeister, F. Arch. Exp. Pathol. Pharmakol. 1888, 24, 247-260; Paloniemi, H. et al., Langmuir 2006, 22, 74-83; Kovtyukhova, N. I. et al., Journal of Physical Chemistry B 2005, 109 2540 -2545; and Rouse, J. H. et al., Langmuir 2004, 16, 3904-3910). The presence of salt did not result in any obvious morphological differences in the coating after multiple deposition cycles (FIGS. 4f and 4g).

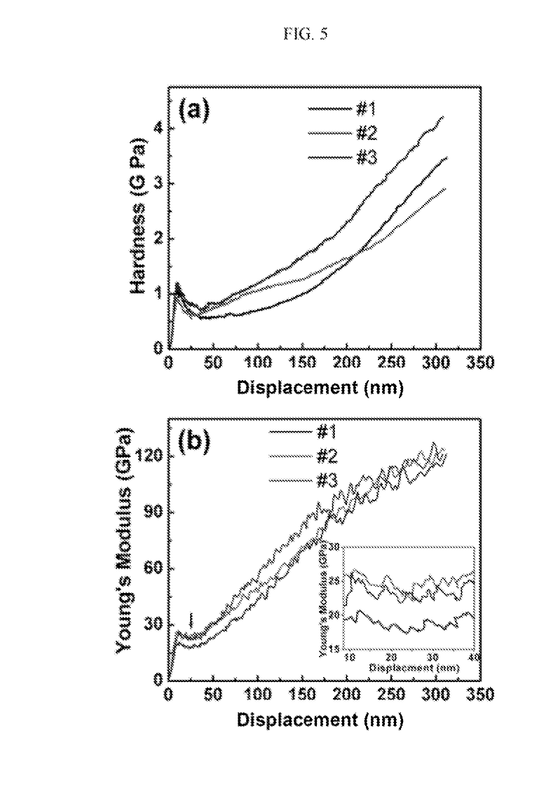

Nanoindentation was used to determine the mechanical properties of 205 nm thick (68 layers) coatings. FIG. 5 shows the hardness and Young's modulus as a function of penetration depth; the hardness was 1 GPa and the Young's Modulus was 22 GPa. These results are similar to those measured by Mamedov et al. (see Mamedov, A. A. et al., Nature Materials 2002, 1, 190-194) and Xue et al. (see Xue, W. et al., Nanotechnology 2007, 18, 14570) and confirm effective load transfer between the SWNTs and the biomacromolecular matrix.

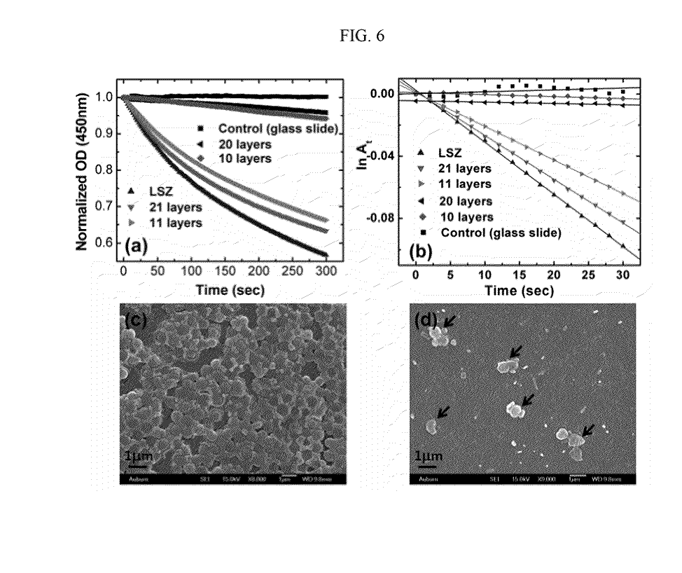

The enzymatic activities of the LBL coatings were evaluated as described in the supporting information. Remarkably, coatings terminating in a LSZ-SWNT layer exhibited a relative antimicrobial activity of 84% compared to 69% in the initial dispersion (FIG. 6). The clearing of the turbid Micrococcus lysodeikticus solution by the coatings is due to the enzyme activity of the exposed LSZ-SWNT layer suggesting a dynamic interaction between the coating surface and the surrounding solution. It is important to note that no lytic activity was observed for surface layers ending with DNA-SWNT or unmodified surfaces (Student's t-test, P<0.05). This confirms that the antimicrobial activity was specifically due to the LSZ enzyme reaction and not the presence of SWNTs. Of particular interest is that the number of layers has an influence on the antimicrobial activity of the coating (FIG. 6b). This behavior suggests that a zone-model behavior is observed as outlined by Ladam et al. (see Ladam, G. et al., Langmuir 2000, 16, 1249-1255) where the substrate affects the growth of initial layers forming Zone I followed by growth of Zone II and Zone III as the number of layers increased. On the other hand, this may also arises from interplay of charges during film growth as the underlying layers are overcompensated compared to terminal layers. (see Schwarz, B. et al., Colloids and Surfaces A: Physicochemical and Engineering Aspects 2002, 198-200, 293-304). This unequal distribution of charges on different layers may influence the activity of LSZ since it must have an optimal balance of charges in order to express lytic activity (see Losso, J. et al., Natural Food Antimicrobial Systems; Naidu, A. S., Ed.; CRS Press: 2000, p 185-210). The coatings exhibited impressive long term stability; no leaching of enzyme was observed in the supernatant when the coatings were stored in buffer and the antimicrobial activity is retained for at least 60 days (see supporting information).

Exposing surfaces to freshly prepared staphylococcus aureus provided further evidence of the antimicrobial activity of LSZ-SWNT terminated coatings. When silicon substrates with and without the LBL assembled coating were incubated with staphylococcus aureus for 24 h at 37° C. and imaged under SEM, significantly more bacteria adhered to the uncoated (FIG. 6c) surface than the coated surface (FIG. 6d). In addition, the few bacteria that adhered to the coated substrate underwent severe morphological changes. In contrast, on the uncoated silicon surface the cells remained intact and maintained their cocci structure. These morphological changes in staphylococcus aureus cells are speculated to be the result of lysozyme triggered autolysis of bacteria which is the generally accepted mechanism of lysozyme action on staphylococcus aureus cells (see Virgilio, R. et al., Journal of Bacteriology 1966, 91, 2018-2024; and Wecke, J. et al., Archives of Microbiology 1982, 131, 116-123).

In conclusion, we have developed a unique multifunctional biomimetic material comprised of SWNT, DNA and LSZ using LBL assembly. Precise control of both layer thickness and SWNT alignment within each layer was achieved, and the final coatings had robust mechanical properties. Coatings ending in an exposed LSZ-SWNT layer exhibit excellent long-term antimicrobial activity. This has several distinct advantages over coatings which release antimicrobials over time; controlled release coatings lose their antimicrobial efficiency once the concentration of the antimicrobial agent drops below the minimum inhibitory concentration (MIC). On the other hand, our non-leaching coatings exhibit robust mechanical properties and long term protection against bacterial colonization. Furthermore, the spectrum of disinfection of LSZ-SWNT layers can be extended to gram-negative bacteria by simply including chelators such as EDTA (see Losso, J. et al., Natural Food Antimicrobial Systems; Naidu, A. S., Ed.; CRS Press: 2000, p 185-210). The results of this research demonstrate the significant possibilities for the molecular design of hybrid structural materials from SWNTs and natural biopolymers. Such robust, antimicrobial materials have significant promise in applications including medicine, aerospace engineering, public transportation, home appliances and sporting goods.

Experimental Section

HiPco SWNT (Rice University) were purified by a thermal oxidation-acid extraction cycle (see Xue, W.; Cui, T. Nanotechnology 2007, 18, 145709). Lysozyme (LSZ) (Hen egg white) and DNA (Calf thymus) were obtained from Sigma and used as received. Microscopy glass slides (Fisher), silicon wafers (Nova electronic material) and freshly cleaved mica were used as substrate materials. Dispersion of SWNT in LSZ and DNA were achieved by the previously published method (Nepal, D. et al., Small 2006, 2, 406-412). In a typical experiment, a solution (1 mg/ml) of LSZ or DNA was mixed with SWNT powder to yield a 0.3 mg/ml concentration of SWNTs in the mixture, followed by sonication for 30 min in an ice bath using a standard probe (13 mm diameter) to obtain a fine black dispersion. The final products of the dispersion were collected from the supernatant employing ultracentrifugation 18,000 g for 3 h. Zeta potential of the prepared dispersion were analyzed using “ZetaPlus” instrument (Brookhaven Instrument Corporation) based on the electrophoretic light scattering (ELS) technique.

Film or Coating Formation: To prepare LBL films or coatings, cationic LSZ-SWNT was alternately assembled with anionic DNA-SWNT. First, glass or silicon slides were cleaned in concentrated H₂SO₄/30% H₂O₂ (3:1) (“Piranha” solution). Then, the slides were immersed alternately in aqueous dispersion of LSZ-SWNT (15 min immersion times) and DNA-SWNT. Doubling of the SWNT deposition time did not affect the results. Therefore, the adsorption time of 15 min was considered sufficient for the formation of a SWNT monolayer. After each layer deposition, the substrate was blown with 50 PSI air from a nozzle for ˜30 sec. Similar technique was employed to SWNT combing (see Shim et al., Langmuir 2005 21, 9381 -9385). Films or coatings were also prepared on evaporated gold surfaces. The in situ assembly of DNA-SWNT & LSZ-SWNT was characterized in real time by SPR using SPREETA™ sensors (Texas Instruments) with two analysis channels. A gold surfaced SPR sensor module, and its supporting hardware and software were coupled to a continuous-flow cell to allow contact with reaction solutions. Experimental setup and cleaning steps were performed as previously described (see Balasubramanian, S. et al., Biosensors and Bioelectronics 2007, 22, (6), 948-955). The sensor was docked with the fluidics block and reference measurements were obtained with air and water as baseline measurements. SPR gold surface was initially modified with diluted alkanethiol solution (11-mercaptoundecanoic acid, 1 mM in absolute ethanol) for 18-24 h followed by electrostatic adsorption of polyethyleneimine (1% in water). Assembly of films or coatings was carried in situ by introducing DNA-SWNT & LSZ-SWNT sequentially to the gold surface. To check the effect of salt, NaCl was added to the dispersion to make 10 mM concentration. Real-time assembly steps were monitored by measuring the change in refractive index (RI) as a function of time followed by integration using SPREETA software. The signal is generally displayed in response unit (RU) (1 Response Unit=10⁻⁶ Refractive index unit).

The thickness and surface coverage of individual layers were calculated assuming ‘linear response regime’ of the evanescent wave, in which the thickness of the adlayers is dₐ<l_{d}, (l_{d} is the decay length of the evanescent wave) (see Jung et al., Langmuir 1998, 14, (19), 5636-5648). Given that the thickness of the individual SWNT, DNA and lysozyme are on the order of few nanometers whereas the decay length of the evanescent wave is on the order of 200 nm, this relationship should hold true. The thickness of the adsorbed layer was calculated using Equation shown below

$$thickness , d = - ( {l}_{decay} / 2 ) * ln ( 1 - ( {n}_{eff} - {n}_{buffer} ) / ( {n}_{adlayer} - {n}_{buffer} ) )$$

$$where , {l}_{decay} = ( λ / 2 π ) * \sqrt{\frac{- 1 * {( {n}_{eff} )}^{4}}{{( {n}_{eff} )}^{2} + {E}_{gold}}}$$

$$λ - 840 nm$$

$${n}_{eff} - effective RI of the adlayer , \\ {n}_{b} - RI of the buffer , \\ {n}_{adlayer} - RI of bulk samples$$

Characterization of Films and Coatings: Vis-near-IR spectra were measured with a Varian Cary 5E spectrophotometer. Ellipsometric measurements were made with Autoelles Rudolph research ellipsometer. The analyzing wavelength was 632 nm, the incident angle was 70°, and the polarizer was set at 45°. Au and Si refractive indices were determined from blank samples. The refractive index of SWNT LBL films was approximated as N_{f}=1.540. The surface morphology was monitored by JEOL 7000F FE-SEM with EDX detector after sputter coating the samples with gold. The morphology was also tested using noncontact tapping mode atomic force microscopy (AFM) using a NanoScope III multimode AFM (Digital Instruments, Santa Barbara, Calif.) apparatus. Raman scattering studies were carried out with Renishaw-in Via Reflex (50× objective) at and at 514 nm (laser). To assess the orientation of the SWNTs measurements were conducted with a well centered 50× objective configured in the vertical direction geometry where the polarizer and the analyzer were parallel to each other and at discrete angles between 0 and 90°. The zeta potential (ζ) of the aqueous solution was measured by light scattering (ELS-8000, Photal, Otsuka Electronics, Japan). A commercially available depth-sensing nanoindentation tester (NanoIndenter XP, MTS) was used to characterize the mechanical properties of the SWNT thin films. Hardness and Young's modulus have been derived from the measured load-contact depth curves following the procedure in the literature. The hardness of the indented material is given by the indentation load divided by the projected contact area (area of the contact at the applied load) of the indentation. Young's modulus of the sample can be determined from the elastic contact stiffness S and the contact area. The contact stiffness is defined as the slope of the upper portion of the uploading curve during the initial stage of unloading. A dynamic indentation technique called continuous stiffness measurement (CSM) developed by Oliver and Pharr was used in the indentation tests. During the CSM process, a small amplitude oscillation with relatively high frequency is superimposed on the dc indenter load control signal. Physically, a three-sided pyramid indenter tip is driven perpendicularly into, then out of, the film. To minimize the influence from the substrates, the indentation depth limits on the SWNT thin films were set ˜20% of the film thickness. A series of ten indentations was performed for each sample. A typical indentation experiment consists of four subsequent steps approaching the surface, loading to peak load, holding the indenter at peak load for 5 s, finally unloading completely. The hold step was included to avoid the influence of creep on the unloading characteristics since the unloading curve was used to obtain the elastic modulus of a material.

Antimicrobial Activity, Turbidimetric rate determination: The antimicrobial activity of LSZ-SWNT in solution was characterized using Micrococcus lysodeikticus, a well-studied substrate organism for LSZ. The assay was performed according to the recommended procedure (Sigma L6876, Enzymatic assay for LSZ). Briefly, 0.015% (w/v) of substrate (A₄₅₀ₙₘ of this suspension=0.6 to 0.7) was prepared in 66 mM potassium phosphate buffer, pH 6.24. Mixing 100 μl of LSZ-SWNT with 2.5 ml of freshly prepared cell suspension resulted in decrease in turbidity of the suspension, thus allowing us to continuously monitor the LSZ activity in real time. The activity of the LSZ-SWNT in solution is reported relative to the activity of unmodified lysozyme.

The activity of LSZ-SWNT incorporated in layer-by-layer assembly prepared on a pre-cleaned glass slide (0.5 cm×0.5 cm) was analyzed similarly. All active films (10, 11, 20 & 21 layers) on the glass slide were introduced in to 200 μl cell suspension with brief mixing and activity was monitored over the time. Unmodified glass slide and glass slide modified with LSZ acts as negative and positive control respectively and activity efficiency is reported relative to the positive control. The data from the turbidimetric assay were fitted using first-order reaction kinetics described by the equation,

Aₜ=Aₒe−kt,
where Aₜ=Absorbance at time (t),
Aₒ=initial absorbance at zero time,
−k=rate of exponential death when lnAₜ is plotted against time

TABLE 1
Comparison of enzymatic activity of lysozyme
in solution and in the assembled coating.

| | |
|---|---|
| In solution | Coating |

| | Activity^{a} | Layers | Activity^{a} |
|---|---|---|---|
| LSZ | 27.8 | LSZ | 3.35 |
| LSZ-SWNT | 19.2 | 21 layers | 2.8 |
| DNA-SWNT | −0.34 | 20 layers | 0.0852 |
| Control^{b} | −0.28 | 11 layers | 2.16 |
| | | 10 layers | 0.128 |
| | | Control^{b} | −0.12 |

^{a}Activity = −k/0.001,
^{b}For solution, phosphate buffer is used as control and for the surface, uncoated glass slide is used as control

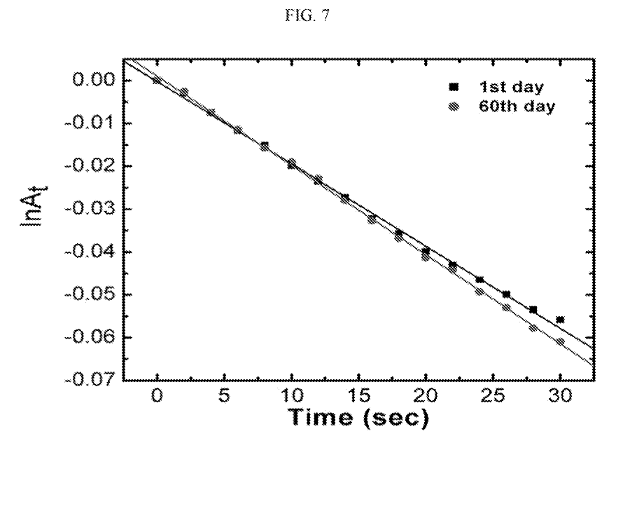

Retention of antimicrobial activity was evaluated after storing the coated slide for 60 days at room temperature (FIG. 7). No significant change was observed. The ability of the film to retain LSZ was further tested by measuring the activity of the liquid medium surrounding the LBL coating (21 layers). The LBL coated glass slide was immersed in 5 ml phosphate buffer for 1 hr (with and without shaking) and 100 μl of this solution was used in turbidimetric assay.

For staphylococcus aureus studies, the bacteria were cultivated in a NZY nutrient broth by shaking at 37° C. for 18 h at 200 rpm. The overnight culture was washed and centrifuged to remove the medium and reconstituted in sterile phosphate buffer solution (PBS) in order to obtain a final concentration of 1.5−3×10⁵ colony forming units (CFU) per ml. Two pieces of each test specimen (Si coated with DNA-SWNT as the top layer, and Si coated with LSZ-SWNT as the top layer) with a fixed surface area of 0.5 cm² were transferred into pre cleaned glass vials containing 5 ml of bacteria in PBS. The samples were incubated at 37° C. for 24 hr before examining under the scanning electron microscope.

### REFERENCES

(1) Wilson, J. W.; Ott, C. M.; zu Bentrup, K. H.; Ramamurthy, R.; Quick, L.; Porwollik, S.; Cheng, P.; McClelland, M.; Tsaprailis, G.; Radabaugh, T.; Hunt, A.; Fernandez, D.; Richter, E.; Shah, M.; Kilcoyne, M.; Joshi, L.; Nelman-Gonzalez, M.; Hing, S.; Parra, M.; Dumars, P.; Norwood, K.; Bober, R.; Devich, J.; Ruggles, A.; Goulart, C.; Rupert, M.; Stodieck, L.; Stafford, P.; Catella, L.; Schurr, M. J.; Buchanan, K.; Morici, L.; McCracken, J.; Allen, P.; Baker-Coleman, C.; Hammond, T.; Vogel, J.; Nelson, R.; Pierson, D. L.; Stefanyshyn-Piper, H. M.; Nickerson, C. A. Proceedings of the National Academy of Sciences 2007, 104, 16299-16304.

(2) Darouiche, R. O. The New England Journal of Internal Medicine 2004, 350, 1422-1429.

(3) Cheng, V. C. C.; Lau, S. K. P.; Woo, P. C. Y.; Yuen, K. Y. Proceedings of the National Academy of Sciences 2007, 20, 660-694.

(4) Chu, C.-M.; Cheng, V. C. C.; Hung, I. F. N.; Chan, K.-S.; Tang, B. S. F.; Tsang, T. H. F.; Chan, K.-H.; Yuen, K.-Y. Emerging Infectious Diseases 2005, 11, 1882-1886.

(5) Klevens, R. M.; Morrison, M. A.; Nadle, J.; Petit, S.; Gershman, K.; Ray, S.; Harrison, L. H.; Lynfield, R.; Dumyati, G.; Townes, J. M.; Craig, A. S.; Zell, E. R.; Fosheim, G. E.; McDougal, L. K.; Carey, R. B.; Fridkin, S. K. The Journal of the American Medical Association 2007, 298, 1763-1771.

(6) Jolles, J.; Jolles, P. Mol. Cell. Biochem. 1984, 63, 165-189.

(7) Proctor, V. A.; Cunningham, F. E. Critical Reviews in Food Science and Nutrition 1988, 26, 359-395.

(8) Losso, J.; Nakai, S.; Charter, E. In Natural Food Antimicrobial Systems; Naidu, A. S., Ed.; CRS Press: 2000, p 185-210.

(9) Huang, J. Y.; Chen, S.; Wang, Z. Q.; Kempa, K.; Wang, Y. M.; Jo, S. H.; Chen, G.; Dresselhaus, M. S.; Ren, Z. F. Nature 2006, 439, 281-281.

(10) O'Connell, M. J.; Bachilo, S. M.; Huffman, C. B.; Moore, V. C.; Strano, M. S.; Haroz, E. H.; Rialon, K. L.; Boul, P. J.; Noon, W. H.; Kittrell, C.; Ma, J.; Hauge, R. H.; Weisman, R. B.; Smalley, R. E. Science 2002, 297, 593-596.

(11) Nepal, D.; Geckeler, K. E. In Functionalization of Carbon Nanotubes Kurt E. Geckeler, Rosenberg, E., Eds.; American Scientific Publishers: Valencia, 2006, p 57-79.

(12) Nepal, D.; Sohn, J.-I.; Aicher, W. K.; Lee, S.; Geckeler, K. E. Biomacromolecules 2005, 6 2919 -2922.

(13) Davis, V. A.; Ericson, L. M.; Parra-Vasquez, A. N.; Fan, H.; Wang, Y.; Prieto, V.; Longoria, J. A.; Ramesh, S.; Saini, R.; Kittrell, C.; Billups, W. E.; Adams, W. W.; Hauge, R. H.; Smalley, R. E.; Pasquali, M. Macromolecules 2004, 37, 154-160.

(14) Rai, P. K.; Pinnick, R. A.; Parra-Vasquez, A. N. G.; Davis, V. A.; Schmidt, H. K.; Hauge, R. H.; Smalley, R. E.; Pasquali, M. J. Am. Chem. Soc. 2006, 128, 591-595.

(15) Barisci, J. N.; Tahhan, M.; Wallace, G. G.; Badaire, S.; Vaugien, T.; Maugey, M.; Poulin, P. Advanced Functional Materials 2004, 14, 133-138.

(16) Nepal, D.; Geckeler, K. E. Small 2006, 2, 406-412.

(17) Nepal, D.; Geckeler, K. E. Small 2007, 3, 1259-1265.

(18) Hammond, P. T. Current Opinion in Colloid & Interface Science 1999, 4, 430-442.

(19) Tang, Z.; Wang, Y.; Podsiadlo, P.; Kotov, N. A. Advanced Materials 2006, 18, 3203-3224.

(20) Jiang, C.; Tsukruk, V. V. Advanced Materials 2006, 18, 829-840.

(21) Zheng, M.; Jagota, A.; Semke, E. D.; Diner, B. A.; Mclean, R. S.; Lustig, S. R.; Richardson, R. E.; Tassi, N. G. Nature Materials 2003, 2, 338-342

(22) Jorio, A.; Dresselhaus, G.; Dresselhaus, M. S.; Souza, M.; Dantas, M. S. S.; Pimenta, M. A.; Rao, A. M.; Saito, R.; Liu, C.; Cheng, H. M. Physical Review Letters 2000, 85, 2617.

(23) Ericson, L. M.; Fan, H.; Peng, H.; Davis, V. A.; Zhou, W.; Sulpizio, J.; Wang, Y.; Booker, R.; Vavro, J.; Guthy, C.; Parra-Vasquez, A. N. G.; Kim, M. J.; Ramesh, S.; Saini, R. K.; Kittrell, C.; Lavin, G.; Schmidt, H.; Adams, W. W.; Billups, W. E.; Pasquali, M.; Hwang, W.-F.; Hauge, R. H.; Fischer, J. E.; Smalley, R. E. Science 2004, 305, 1447-1450.

(24) Chae, H. G.; Minus, M. L.; Kumar, S. Polymer 2006, 47, 3494-3504.

(25) Doi, M.; Edwards, S. F. The Theory of Polymer Dynamics; Oxford University Press: Oxford, 1986.

(26) Duggal, R.; Pasquali, M. Physical Review Letters 2006, 96.

(27) Shim, B. S.; Kotov, N. A. Langmuir 2005 21, 9381 -9385.

(28) Hedberg, J.; Dong, L.; Jiao, J. Applied Physics Letters 2005, 86, 143111-3.

(29) Davis, V. A.; Pasquali, M. In Nanoengineering of Structural, Functional and Smart Materials; Schulz, M. J., Kelkar, A., Sundaresan, M. J., Eds.; CRC Press: Boca Raton, 2005.

(30) Hofmeister, F. Arch. Exp. Pathol. Pharmakol. 1888, 24, 247-260.

(31) Paloniemi, H.; Lukkarinen, M.; Aaritalo, T.; Areva, S.; Leiro, J.; Heinonen, M.; Haapakka, K.; Lukkari, J. Langmuir 2006, 22, 74-83.

(32) Kovtyukhova, N. I.; Mallouk, T. E. Journal of Physical Chemistry B 2005, 109 2540 -2545.

(33) Rouse, J. H.; Lillehei, P. T.; Sanderson, J.; Siochi, E. J. Langmuir 2004, 16, 3904-3910.

(34) Mamedov, A. A.; Kotov, N. A.; Prato, M.; Guldi, D. M.; Wicksted, J. P.; Hirsch, A. Nature Materials 2002, 1, 190-194.

(35) Xue, W.; Cui, T. Nanotechnology 2007, 18, 145709.

(36) Ladam, G.; Schaad, P.; Voegel, J. C.; Schaaf, P.; Decher, G.; Cuisinier, F. Langmuir 2000,16, 1249-1255.

(37) Schwarz, B.; Schonhoff, M. Colloids and Surfaces A: Physicochemical and Engineering Aspects 2002, 198-200, 293-304.

(38) Virgilio, R.; Gonzalez, C.; Munoz, N.; Mendoza, S. Journal of Bacteriology 1966, 91, 2018-2024.

(39) Wecke, J.; Lahav, M.; Ginsburg, I.; Giesbrecht, P. Archives of Microbiology 1982, 131, 116-123.

(40) Xu, Y. et al., Nano Letters 2005, 5, (1), 163-168.

(41) Balasubramanian, S.; Sorokulova, I. B.; Vodyanoy, V. J.; Simonian, A. L., Lytic phage as a specific and selective probe for detection of Staphylococcus aureus—A surface plasmon resonance spectroscopic study. Biosensors and Bioelectronics 2007, 22, (6), 948-955.

(42) Jung, L. S.; Campbell, C. T.; Chinowsky, T. M.; Mar, M. N.; Yee, S. S., Quantitative Interpretation of the Response of Surface Plasmon Resonance Sensors to Adsorbed Films. Langmuir 1998, 14, (19), 5636-5648.

(43) Bhushan, B.; Li, X., Nanomechanical characterization of solid surfaces and thin films. International Materials Reviews 2003, 48, 125-164.

In the foregoing description, certain terms have been used for brevity, clearness, and understanding. No unnecessary limitations are to be implied therefrom beyond the requirement of the prior art because such terms are used for descriptive purposes and are intended to be broadly construed. The different configurations, systems, and method steps described herein may be used alone or in combination with other configurations, systems, and method steps. It is to be expected that various equivalents, alternatives and modifications are possible within the scope of the appended claims. The aforementioned references are incorporated herein by reference in their entireties.

We claim:

1. A carbon nanocomposite film comprising multiple layers, wherein the multiple layers comprise biomolecules bound to aligned carbon nanotubes and the multiple layers individually have an average thickness of about 1-2 times average diameter of the carbon nanotubes.

2. The film of claim 1, wherein the carbon nanotubes are single-walled carbon nanotubes and the multiple layers individually have an average thickness of about 1-2 nm.

3. The film of claim 1, wherein the biomolecules are selected from a group consisting of polypeptides, polynucleotides, or a mixture thereof.

4. The film of claim 1, wherein the biomolecules are polypeptides.

5. The film of claim 4, wherein the polypeptides are anti-bacterial polypeptides and the film has anti-bacterial activity.

6. The film of claim 5, wherein the polypeptides are lysozyme molecules and the film has lysozyme activity.

7. The film of claim 4, wherein the polypeptides are organophosphorus hydrolase molecules and the film has organophosphorus hydrolase activity.

8. The film of claim 4, wherein the biomolecules are polynucleotides.

9. The film of claim 1, wherein the multiple layers comprise:
(a) at least a first layer wherein the nanotubes are aligned in a first direction; and
(b) at least a second layer adjacent to the first layer wherein the nanotubes are aligned in a second direction;
the first direction and the second direction being parallel.

10. The film of claim 1, wherein the multiple layers comprise:
(a) at least a first layer wherein the nanotubes are aligned in a first direction; and
(b) at least a second layer adjacent to the first layer wherein the nanotubes are aligned in a second direction;
the first direction and the second direction being non-parallel.

11. The film of claim 1, wherein the multiple layers comprise:
(a) at least a first layer wherein the nanotubes are aligned in a first direction; and
(b) at least a second layer adjacent to the first layer wherein the nanotubes are aligned in a second direction;
the first direction and the second direction being at a 45° angle.

12. The film of claim 1, wherein the multiple layers comprise:
(a) at least a first layer wherein the nanotubes are aligned in a first direction; and
(b) at least a second layer adjacent to the first layer wherein the nanotubes are aligned in a second direction;
the first direction and the second direction being perpendicular.

13. The film of claim 12, wherein the nanotubes of each layer of the multiple layers are aligned perpendicularly to the nanotubes of each adjacent layer.

14. The film of claim 1, wherein the multiple layers comprise:
(a) at least a first layer comprising positively-charged polypeptides bound to single wall carbon nanotubes; and
(b) at least a second layer adjacent to the first layer, the second layer comprising negatively-charged polymers bound to single wall carbon nanotubes.

15. The film of claim 1, wherein the multiple layers comprise:
(c) at least a first layer comprising negatively-charged polypeptides bound to single wall carbon nanotubes; and
(d) at least a second layer adjacent to the first layer, the second layer comprising positively-charged polymers bound to single wall carbon nanotubes.

16. The film of claim 1, wherein the film has a thickness of at least about 5 nm.

17. The film of claim 1, wherein the film has a hardness of at least about 0.5 GPa.

18. The film of claim 1, wherein the film has a Young's modulus of at least about 10 GPa.

19. The film of claim 1 bound to a solid substrate.

20. A method for preparing a coated substrate using a layer-by-layer technique, the method comprising:
(a) coating the substrate with a first layer of biomolecules bound to carbon nanotubes and aligning the carbon nanotubes, wherein the first layer has a thickness of about 1-2 nm and the first layer has a surface charge that is opposite to a surface charge for the substrate;
(b) subsequently coating the substrate with a second layer of biomolecules bound to carbon nanotubes and aligning the carbon nanotubes, wherein the second layer has a thickness of about 1-2 nm and the second layer has a surface charge that is opposite to the surface charge for the first layer; and
(c) repeating (a) and (b) to provide a coating on the substrate having a thickness of at least about 50 nm.

21. The method of claim 20, wherein the carbon nanotubes are aligned by applying shear force.

22. The method of claim 20, wherein the biomolecules of at least one of the first layer and the second layer are anti-bacterial polypeptides.

23. A method of killing bacteria, the method comprising contacting the bacteria with a carbon nanocomposite film comprising multiple layers, wherein the multiple layers comprise anti-bacterial polypeptides bound to aligned carbon nanotubes and the multiple layers individually have an average thickness of about 1-2 times average diameter of the carbon nanotubes.

24. A method of hydrolyzing organophosphorus compounds, the method comprising contacting the compounds with a carbon nanocomposite film comprising multiple layers, wherein the multiple layers comprise organophosphorus hydrolase polypeptides bound to aligned carbon nanotubes and the multiple layers individually have an average thickness of about 1-2 times average diameter of the carbon nanotubes.
